(19) United States
(12) Patent Application Publication
(10) Pub. No.: US 2021/0130409 A1
(43) Pub. Date: May 6, 2021

(54) Method for the Solid-Phase Synthesis of Cyclic Pentapeptides

(71) Applicant: Alsonex Pty Ltd, Brisbane (AU)

(72) Inventors: Jiang Zhengguo, Guangzhou City (CN); Forni Luciano, La Louviere (BE); Alan Robertson, Brisbane (AU)

(21) Appl. No.: 16/639,414

(22) PCT Filed: Aug. 16, 2018
(86) PCT No.: PCT/AU2018/050872

§ 371 Date: Feb. 14, 2020

(30) Foreign Application Priority Data
Sep. 1, 2017 (AU) 2017903543

(51) Int. Cl. C07K 7/56; C07K 1/06

(52) U.S. Cl. CPC C07K 7/56; C07K 1/061

(57) ABSTRACT

A method for the synthesis of a cyclic ornithine-proline-D-cyclohexylalanine-tryptophan-arginine pentapeptide of Formula A;

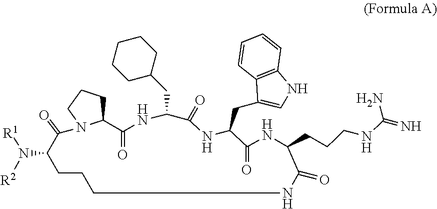

wherein R¹ and R² are, independently, —H, or —C(O)R³ where R³ is —CH₂Ph, —CH₂CH₂Ph, —CH═CHPh, —C(NHAC)CH₂Ph;
   the method comprising the steps of;
   forming a linear proline-D-cyclohexylalanine-tryptophan-arginine-ornithine pentapeptide of Formula B, attached to a polymeric resin;

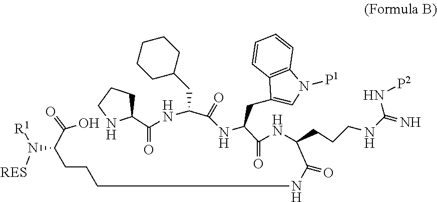

wherein R¹ is as for Formula A, RES indicates the polymeric resin, and P¹ and P² are protecting groups;
   cyclising the linear pentapeptide of Formula B to form a cyclic pentapeptide of Formula C, attached to the polymeric resin;

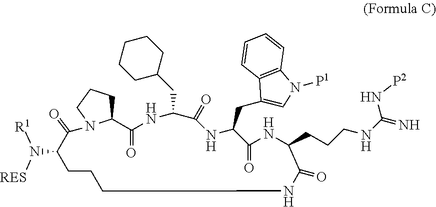

cleaving the cyclic peptide of Formula C from the resin providing a cleaved cyclic pentapeptide having a free amine group of an ornithine residue;
optionally substituting the free amine group of the ornithine residue of the cleaved cyclic peptide;
removing the protecting groups P¹ and P², to provide the cyclic peptide of Formula A.

### TECHNICAL FIELD

The present invention relates to a method for the synthesis of certain cyclic peptides. In particular, the present invention relates to a method for the preparation of cyclic pentapetides incorporating the cyclic pentapeptide structure c[ornithine-proline-D cyclohexylalanine-tryptophan-arginine], including the acylated cyclic pentapetides HCin-[ornithine-proline-D cyclohexylalanine-tryptophan-arginine] (PMX205) and AcPhe-[ornithine-proline-D cyclohexylalanine-tryptophan-arginine] (PMX53), which are macrocyclic peptidomimetics of the human plasma protein C5a.

### BACKGROUND ART

The following discussion of the background art is intended to facilitate an understanding of the present invention only. The discussion is not an acknowledgement or admission that any of the material referred to in the discussion is, or was part of, the common general knowledge as at the priority date of the application.

Inflammation plays a major role in the progression of neurodegenerative diseases such as amyotrophic lateral sclerosis (Woodruff T M, Constantini K J, Crane J W, et al. The complement factor C5a contributes to pathology in a rat model of amyotrophic lateral sclerosis. J Immunol. 2008; 181:8727-8734) and Alzheimer's disease (Fonseca M L, Ager R R, Chu S-H, et al. Treatment with a C5aR antagonist decreases pathology and enhances behavioral performance in murine models of Alzheimer's disease, J Immunol. 2009; 183:1375-1383; Ager R R, Fonseca M L, Chu S-H, et al. Microglial C5aR (CD88) expression correlates with amyloid-β deposition in murine models of Alzheimer's disease, J Neurochem. 2010; 113:389-401). Additionally, complement activation, has been implicated in the progression of asthma (Elizabeth B. Staab E B, Sanderson S D, Wells S M, et al. Treatment with the C5a receptor/CD88 antagonist PMX205 reduces inflammation in a murine model of allergic asthma, International Immunopharmacology 2014, 21:293-300 Inhibition of the major complement receptor C5aR1 results in a significant reduction of pathology in rodent models of these conditions. The cyclic hexapeptide-based compounds PMX53 (AcPhe-[ornithine-proline-D cyclohexylalanine-tryptophan-arginine]) and PMX205 (HCin-[ornithine-proline-D cyclohexylalanine-tryptophan-arginine]) are C5a receptor-1 antagonists.

A solution phase synthesis of PMX53 is described in published United States Patent Application 20070249526 (Abbenante et al.). However, the procedure requires a highly dilute solution phase cyclisation of the linear peptide that is accompanied by up to 4% racemisation and a significant amount of polymeric by product that is difficult to remove. The reported yield of the cyclisation is 33%. The synthesis of PMX205 is described in Milton et al. (Improving the Fmoc Solid Phase Synthesis of the Cyclic Hexapeptide Complement C5a Antagonist, PMX205, Int J Pept Res Ther. 2011 December; 17(4): 337-342. doi:10.1007/s10989-011-9273-9). Milton et al. report that the cyclisation step is undertaken at a concentration of 0.0005 M in dimethyl formamide and that the reaction takes four days to complete. While the reported yield of the cyclisation step is 82%, the extremely large volume of solvent that would be required for cyclisation of commercial quantities of PMX205 renders this approach impractical on an industrial scale.

Furthermore, the data reported by Milton et al. are indicative of racemisation during the cyclisation reaction analogous to that reported in US Patent Application 20070249526 (Abbenante et al.). Although racemisation during cyclisation is not explicitly discussed by Milton et al., the analytical C4 RP-HPLC on purified PMX205 (see FIG. 4) prepared using the method described by Milton et al. shows a front running impurity that is indicative of racemisation having taken pace.

To enable the synthesis of commercial quantities of these and other C5a Receptor-1 antagonists, an alternative approach is required. Advantageously, any such approach should avoid highly dilute cyclisation conditions and reduce racemization.

It is against this background that the present invention has been developed.

The present invention seeks to overcome, or at least ameliorate, one or more of the deficiencies of the prior art mentioned above, or to provide the consumer with a useful or commercial choice.

### SUMMARY OF INVENTION

In accordance with the present invention, there is provided a method for the synthesis of a cyclic ornithine-proline-D-cyclohexylalanine-tryptophan-arginine pentapeptide of Formula A;

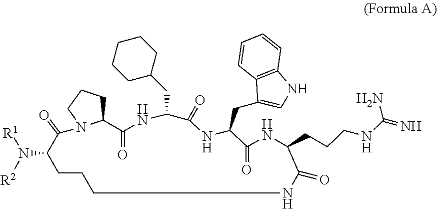

wherein R¹ and R² are, independently, —H, or —(O)R³ where R³ is —CH₂Ph, —CH₂CH₂Ph, —CH═CHPh, —C(NHAC)CH₂Ph;
   the method comprising the steps of;
   forming a linear proline-D-cyclohexylalanine-tryptophan-arginine-ornithine pentapeptide of Formula B, attached to a polymeric resin;

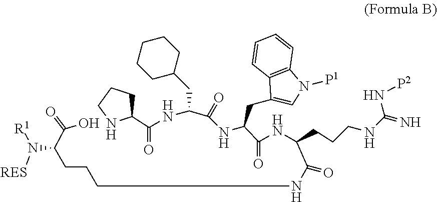

wherein R¹ is as for Formula A, RES indicates the polymeric resin, and P¹ and P² are protecting groups;
   cyclising the linear pentapeptide of Formula B to form a cyclic pentapeptide of Formula C, attached to the polymeric resin;

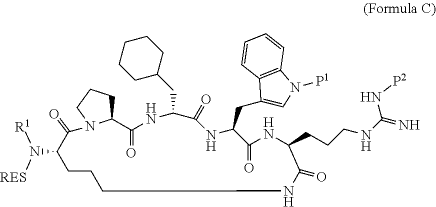

cleaving the cyclic peptide of Formula C from the resin providing a cleaved cyclic pentapeptide having a free amine group of an ornithine residue;
optionally substituting the free amine group of the ornithine residue of the cleaved cyclic peptide;
removing the protecting groups P¹ and P², to provide the cyclic peptide of Formula A.

By cyclising the linear pentapeptide on the resin, the method of the present invention substantially obviates the need to undertake the cyclisation reaction at high dilutions, as is typically required when undertaking cyclisation reactions in solution. High dilutions are typically necessary to favour cyclisation of a peptide, rather than the joining of peptides to form linear peptides. At high dilutions, there is less frequent contact between separate peptide molecules. When considering commercial quantities of cyclic peptides, high dilutions mean high solvent volumes to the extent that the approach may be impractical.

Furthermore, the inventors have observed that the on-resin cyclisation via the N-terminus of the proline residue and the C-terminus of ornithine proceeds in such a manner that a low level of racemisation is observed. For example, the level of racemisation observed in embodiments of the invention is lower than that indicated in the disclosure by Milton et al and Abbenante et al., to which the inventors refer in the discussion of the background to the invention.

### DESCRIPTION OF EMBODIMENTS

As noted in the summary of the invention, in accordance with the present invention, there is provided a method for the synthesis of a cyclic ornithine-proline-D-cyclohexylalanine-tryptophan-arginine pentapeptide of Formula A;

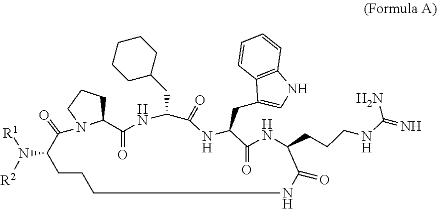

wherein R¹ and R² are, independently, —H, or —(O)R³ where R³ is —CH₂Ph, —CH₂CH₂Ph, —CH═CHPh, —C(NHAC)CH₂Ph;
   the method comprising the steps of;
   forming a linear proline-D-cyclohexylalanine-tryptophan-arginine-ornithine pentapeptide of Formula B, attached to a polymeric resin;

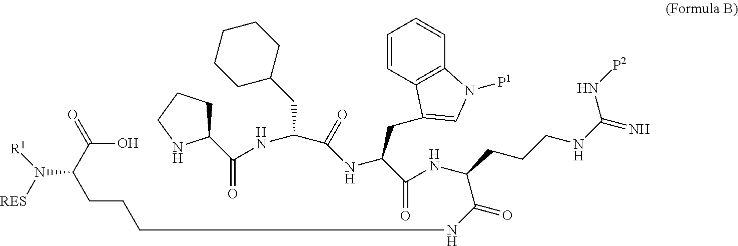

wherein R¹ is as for Formula A, RES indicates the polymeric resin, and P¹ and P² are protecting groups;
   cyclising the linear pentapeptide of Formula B to form a cyclic pentapeptide of Formula C, attached to the polymeric resin;

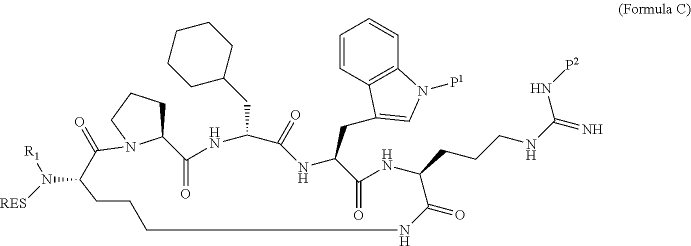

cleaving the cyclic peptide of Formula C from the resin providing a cleaved cyclic pentapeptide having a free amine group of an ornithine residue;
optionally substituting the free amine group of the ornithine residue of the cleaved cyclic peptide;
removing the protecting groups P¹ and P², to provide the cyclic peptide of Formula A.

In a preferred form of the invention, the step of cyclising the linear pentapeptide of Formula B to form a cyclic pentapeptide of Formula C, attached to the polymeric resin comprises the steps of treating the linear pentapeptide of Formula B with a combination of a coupling agent and a base.

While it will be understood that any known coupling agent and base could be used in the step of the step of cyclising the linear pentapeptide of Formula B to form a cyclic pentapeptide of Formula C, such as (benzotriazol-1-yloxy)tris(dimethylamino)phosphonium hexafluorophosphate (BOP) in the presence of a base such as diisopropylethylamine (DIPEA), NaHCO₃ or tetramethylethylenediamine (TMEDA), the combinations of O-[(ethoxycarbonyl)cyanomethylenamino]-N,N,N′,N′-tetramethyluronium tetrafluoroborate (TOTU) with DIPEA as the base and benzotriazol-1-yl-oxytripyrrolidinophosphonium hexafluorophosphate (PyBOP) and DIPEA as the base was found to be advantageous.

In certain forms of the invention, the step of cyclising the linear pentapeptide of Formula B to form a cyclic pentapeptide of Formula C, attached to the polymeric resin, may comprise the step of treating the linear pentapeptide of Formula B with a solution of O-[(ethoxycarbonyl)cyanomethylenamino]-N,N,N′,N′-tetramethyluronium tetrafluoroborate (TOTU) and diisopropylethylamine (DIPEA) in dimethylformamide.

In certain forms of the invention, the step of cyclising the linear pentapeptide of Formula B to form a cyclic pentapeptide of Formula C, attached to the polymeric resin, may comprise the step of treating the linear pentapeptide of Formula B with a solution of benzotriazol-1-yl-oxytripyrrolidinophosphonium hexafluorophosphate (PyBOP) and diisopropylethylamine (DIPEA) in dimethylformamide.

### Protected Ornithine Intermediate

In a preferred form, the step of forming a linear peptide of Formula B, attached to a polymeric resin; comprises the step of forming the intermediate compound of Formula D;

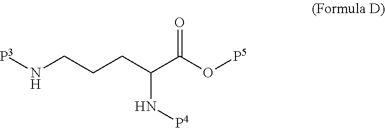

wherein P³, P⁴ and P5 are protecting groups.

In a preferred form, the step of forming a linear peptide of Formula B, attached to a polymeric resin; comprises the step of forming the intermediate compound of Formula D;

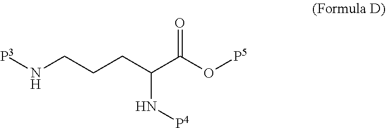

wherein P³ and P⁴ are, independently, selected from the group: 9-fluorenylmethyl carbamate (Fmoc), 2,2,2-trichloroethyl carbamate (Troc), t-butyl carbamate (Boc), allyl carbamate (Alloc), 2-trimethylsilylethyl (Teoc) and benzyl carbamate (Cbz);
P⁵ is selected from the group: -Me, -Et, -tBu, -Bz, —CH₂CH═CH (Alloc).

In preferred forms of the invention, P³ and P⁴ are, independently, selected from the group: Fmoc and Boc.

Techniques for the esterification of a carboxylic acid to produce an ester protecting group of P⁵ are well known to persons skilled in the art. See for example, March's Advanced Organic Chemistry: Reactions Mechanisms and Structure, 5^{th} Edition, Michael B. Smith and Jerry March, Wiley-Interscience at, for example, pp 484-486.

In a preferred form of the invention, the compound of Formula D is produced by reacting a compound of Formula E:

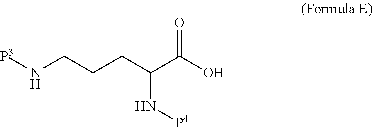

with a compound of the formula P⁵-X, where X is a halide or an alcohol.

In a preferred form of the invention, the method comprises the step of coupling the compound of Formula D to a polymeric resin to produce a compound of Formula F:

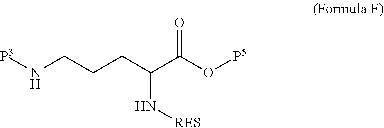

In a preferred form of the invention, P⁵ is Alloc.

In a preferred form of the invention, P³ is Fmoc.

Where P⁵ is Alloc, and the compound of Formula D is produced by reacting a compound of Formula E with a compound of the formula X—CH₂CH═CH, in a preferred form of the invention, the compound of formula X—CH₂CH═CH is 3-bromopropene.

In a preferred form of the invention, the polymeric resin comprises a linker, wherein the linker capable of coupling to an amine group of an amino acid.

In a preferred form of the invention, the polymeric resin comprises a linker, wherein the linker is selected from the group: trityl, 2-chlorotrityl chloride, alkoxybenzyl alcohol (such as Wang resin).

In a preferred form of the invention, the linker is 2-chlorotrityl chloride.

### On-Resin Peptide Elongation

In a preferred form of the invention, after the steps of producing the Compound of Formula D, and coupling the Compound of Formula D to the polymeric resin to produce the compound of Formula F, the method comprises the step of sequentially coupling the following amino acids commencing at the distal amine of ornithine;
1) arginine;
2) tryptophan;
3) D-cyclohexylalanine; and
4) proline;
   to produce the linear pentapeptide of Formula B.

The coupling reactions may be performed using any suitable known technique, preferably involving a coupling agent and a base such as BOP and diphenylphosphonylazide (DPPA).

Although the sequential coupling of amino acids in the manner described above is preferred, the present invention encompasses obvious variations where appropriate combinations of up to four of amino acids are coupled prior to coupling with the distal amine of ornithine.

For example, the present invention encompasses methods where the tetrapeptide [Pro-D-Cha-Trp-Arg] is assembled, then the C-terminus of Arg coupled with distal amine of ornithine to form the polymeric resin attached pentapeptide [Orn-Pro-D-Cha-Trp-Arg] of Formula B as described above.

As would be understood by a person skilled in the art, solid phase peptide synthesis involves a sequence of protecting the N terminus of the amino acid to be added, coupling the C terminus of the protected amino acid to form an amide group of the amino acid attached to the resin, de-protecting the N terminus of the newly coupled amino acid, and repeating the cycle. Various protection and de-protection systems are known to those skilled in the art, including those based on tertiary butylcarbonyl groups (Boc, also known as tBoc), removed under moderately strong acidic conditions (such as with TFA) and 9-fluorophenylmethoxycrabonyl groups (Fmoc), removed under mild basic conditions.

Preferably the N-protecting group is a carbamate such as, 9-fluorenylmethyl carbamate (Fmoc), 2,2,2-trichloroethyl carbamate (Troc), t-butyl carbamate (Boc), allyl carbamate (Alloc), 2-trimethylsilylethyl (Teoc) and benzyl carbamate (Cbz).

In a preferred form of the present invention, the N-terminus of the amino acid to be coupled is protected by Fmoc, and deprotected after coupling using TFA.

### Side Chain Protection

In preferred forms of the invention, in addition to the N-terminal amine, other functional groups of the amino acids may be protected prior to coupling. The manner in which side groups are protected may be influenced by the protection system used for the N-terminus of the uncoupled amino acid. For example, if Fmoc is used to protect the N-terminus, Boc may be used to protect one other functional groups, and vice versa.

The amino acid side chains may be protected, for example, the carboxyl groups of aspartic acid, glutamic acid and α-aminoadipic acid may be esterified (for example as a C₁-C₆ alkyl ester), the amino groups of lysine, ornithine and 5-hydroxylysine, may be converted to carbamates (for example as a C(═O)OC₁-C₆ alkyl or C(═O)OCH₂Ph carbamate) or imides such as thalimide or succinimide, the hydroxyl groups of 5-hydroxylysine, 4-hydroxyproline, serine, threonine, tyrosine, 3,4-dihydroxyphenylalanine, homoserine, .alpha.-methylserine and thyroxine may be converted to ethers (for example a C₁-C₆ alkyl or a (C₁-C₆ alkyl)phenyl ether) or esters (for example a C═OC₁-C₆ alkyl ester) and the thiol group of cysteine may be converted to thioethers (for example a C₁-C₆ alkyl thioether) or thioesters (for example a C(═O)C₁-C₆ alkyl thioester).

In a preferred form of the present invention, prior to coupling to another amino acid, the distal amine of arginine is protected. Preferably still, the distal amine of arginine is protected with 2,2,4,6,7-pentamethyldihydrobenzofurane-5-sulfonyl (Pbf).

In a preferred form of the present invention, where Fmoc is used to protect the N-terminus prior to coupling, the indole nitrogen of tryptophan is protected prior to coupling. Preferably still, the indole nitrogen of tryptophan is protected by Boc.

### Cyclisation

In a preferred form of the invention, the step of cyclising the linear pentapeptide of Formula B to form a cyclic pentapeptide of Formula C attached to the polymeric resin, comprises the steps of:
deprotecting the C-terminus of the ornithine residue; and
deprotecting the N-terminus of the proline residue;
condensing the C-terminus of the ornithine residue and the N-terminus of the proline residue.

### Cleavage

The manner in which the step of cleaving the cyclic peptide of Formula C from the resin providing a cleaved cyclic pentapeptide having a free amine group of an ornithine residue is dependent on the nature of the linker coupling the compound of Formula C to the resin.

Where the linker is selected from the preferred group of: trityl, 2-chlorotrityl chloride, alkoxybenzyl alcohol (such as Wang resin), the step of cleaving the cyclic peptide of Formula C from the resin providing a cleaved cyclic pentapeptide having a free amine group of an ornithine residue preferably comprises the step of treating the cyclic peptide of Formula C attached to the polymeric resin with a solution of a fluoride-generating agent. In a preferred form of the invention, the fluoride-generating agent is selected from the group: hydrogen fluoride or triflouracetic acid. In a particularly preferred form of the invention, the fluoride-generating agent is trifluoroacetic acid.

### Ornithine Amine Substitution

In preferred forms of the invention, the method comprises the step of substituting the free amine group of the ornithine residue of the cleaved cyclic peptide.

In preferred forms of the invention, the method comprises the step of acylating the free amine group of the ornithine residue of the cleaved cyclic peptide with an acylating agent to provide the cyclic peptide of Formula A.

In one preferred embodiment of the invention, the acylating agent is a 3-phenylpropionyl halide, such as 3-phenylpropionyl chloride, and the compound of Formula A is HCin-[ornithine-proline-D cyclohexylalanine-tryptophan-arginine], also known as PXM-205.

In an alternate preferred embodiment of the invention, the acylating agent is an N-protected-L-phenylalanine, such as N-acetyl-L-phenyl alanine and the compound of Formula A is Ac-Phe-[Orn-Pro-D-Cha-Trp-Arg], also known as PMX53.

Conditions appropriate for acylating an amine group are well known to persons skilled in the art. See, for example Christian A. G. N. Montalbetti and Virginie Falque, Tetrahedron 2005, 61, 10827-10852

### Deprotection

Where the method comprises the step of protecting other functional groups of the amino acids prior to coupling, in a preferred form of the invention, after the step of cleaving the cyclic peptide of Formula C from the resin providing a cleaved cyclic pentapeptide having a free amine group of an ornithine residue, and after any optional step of substituting the free amine group of the ornithine residue of the cleaved cyclic peptide, the method comprises the step of deprotecting said functional groups to produce the compound of Formula A.

In the preferred form of the present invention, where the distal amine of arginine is protected with 2,2,4,6,7-pentamethyldihydrobenzofurane-5-sulfonyl (Pbf), the step of deprotecting said functional groups to produce the compound of Formula A comprises the step of treating the protected peptide with an acid, preferably trifluoroacetic acid.

In the preferred form of the invention, where the distal nitrogen of tryptophan is protected with t-butyloxycarbonyl (tBoc or Boc), the step of deprotecting said functional group comprises the step of treating the protected peptide with an acid, preferably trifluoroacetic acid.

In the preferred form of the invention, where 9-fluorenylmethoxycarbonyl (Fmoc) is used to protect the N-terminus prior to coupling, the step of deprotecting said functional groups to produce the compound of Formula A comprises the step of treating the protected peptide with a base, preferably piperidine.

### Scale

As described above, avoiding significant volumes of solvent means that the method of the present invention can be practically applied on a commercial scale. In a preferred form of the present invention, the method produces in excess of 100 g of the compound of Formula A. In a preferred form of the present invention, the method produces in excess of 200 g of the compound of Formula A. In a preferred form of the present invention, the method produces in excess of 2000 g of the compound of Formula A.

### Derivatives, Analogues and Salts

The present invention also provides salts and solvates of compound produced by methods of the invention.

### Compounds Produced by the Methods of the Invention

The present invention also provides a compound prepared by the method of the invention.

### Pharmaceutical Compositions

The present invention also provides a pharmaceutical composition, comprising a compound produced by a method of the invention optionally together with one or more pharmaceutically acceptable excipients.

Additionally, such a pharmaceutical composition or medicament can comprise a pharmaceutically acceptable carrier, adjuvant, or vehicle. A “pharmaceutically acceptable carrier, adjuvant, or vehicle” according to the invention refers to a non-toxic carrier, adjuvant or vehicle that does not destroy the pharmacological activity or physiological targeting of the modulator with which it is formulated. Pharmaceutically acceptable carriers, adjuvants or vehicles that can be used in the pharmaceutical compositions of this invention include, but are not limited to those that can be applied cranially or intracranially, or that can cross the blood-brain barrier (BBB). Notwithstanding this, the pharmaceutical compositions of the invention can include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the present invention may be administered orally, parenterally, by inhalation spray, topically, rectally, nasally, buccally, vaginally, cerebrally, or via an implanted reservoir.

The term parenteral as used herein includes subcutaneous, intravenous, intramuscular, intra-articular, intra-synovial, intrasternal, intrathecal, intrahepatic, intralesional and intracranial injection or infusion techniques. The pharmaceutical compositions are administered orally, intraperitoneally or intravenously. Sterile injectable forms of the pharmaceutical compositions of this invention may be aqueous or oleaginous suspension. These suspensions can be formulated according to techniques known in the art using suitable dispersing or wetting agents and suspending agents. The sterile injectable preparation can also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

As such, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavouring or colouring agents may also be added.

Alternatively, the pharmaceutical composition as defined herein may be administered in the form of suppository for rectal administration. Such a suppository can be prepared by mixing the agent with a suitable non-irritating excipient that is solid at room temperature but liquid at rectal temperature and, therefore, will melt in the rectum to release the drug. Such materials include cocoa butter, beeswax and polyethylene glycols.

The pharmaceutical composition as defined herein may also be administered topically, especially when the target of treatment includes areas or organs readily accessible by topical application, including diseases of the brain, other intra-cranial tissues, the eye, or the skin. Suitable formulations are readily prepared for each of these areas or organs.

For topical applications, the pharmaceutical composition as defined herein may be formulated in a suitable ointment containing modulators as identified herein, suspended or dissolved in one or more carriers. Carriers for topical administration of the peptide include, but are not limited to, mineral oil, liquid petrolatum, white petrolatum, propylene glycol, polyoxyethylene, polyoxypropylene compound, emulsifying wax and water. Alternatively, the pharmaceutical composition as defined herein can be formulated in a suitable lotion or cream containing the peptide suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

The pharmaceutical composition as defined herein may also be administered by nasal aerosol or inhalation. Such a composition may be prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. The pharmaceutically acceptable composition or medicament herein is formulated for oral or parenteral administration, e.g. by injection.

For treatment purposes, a non-toxic, effective amount of a compound of the invention may be used for preparation of a pharmaceutical composition as defined above. Therefore, an amount of a compound of the invention may be combined with the carrier material(s) to produce a composition as defined above.

The pharmaceutical composition is typically prepared in a single (or multiple) dosage form, which will vary depending upon the host treated and the particular mode of administration. Usually, the pharmaceutical composition is formulated so that a dosage range per dose of 0.0001 to 100 mg/kg body weight/day of a compound of the invention can be administered to a patient receiving the pharmaceutical composition. Preferred dosage ranges per dose vary from 0.01 mg/kg body weight/day to 50 mg/kg body weight/day, even further preferred dosage ranges per dose range from 0.1 mg/kg body weight/day to 10 mg/kg body weight/day.

However, dosage ranges and treatment regimens as mentioned above may be adapted suitably for any particular patient dependent upon a variety of factors, including the activity of the specific modulator employed, the age, body weight, general health, sex, diet, time of administration, rate of excretion, drug combination, the judgment of the treating physician and the severity of the particular disease being treated. In this context, administration may be carried with in an initial dosage range, which may be varied over the time of treatment, e.g. by increasing or decreasing the initial dosage range within the range as set forth above. Alternatively, administration may be carried out in a continuous manner by administering a specific dosage range, thereby maintaining the initial dosage range over the entire time of treatment. Both administration forms may furthermore be combined, e.g. if the dosage range is to be adapted (increased or decreased) between various sessions of the treatment but kept constant within the single session so that dosage ranges of the various sessions differ from each other.

When used therapeutically, a compound of the invention of the invention is administered in therapeutically effective amounts. In general, a therapeutically effective amount means an amount necessary to delay the onset of, inhibit the progression of, or halt altogether the particular condition being treated. Generally, a therapeutically effective amount will vary with the subject's age and condition, as well as the nature and extent of the disease in the subject, all of which can be determined by one of ordinary skill in the art. The dosage may be adjusted by the individual physician, particularly in the event of any complications being experienced.

### DEFINITIONS

Throughout this specification, unless the context requires otherwise, the word “comprise” or variations such as “comprises” or “comprising”, will be understood to imply the inclusion of a stated integer or group of integers but not the exclusion of any other integer or group of integers.

The invention described herein may include one or more range of values (e.g. size, displacement and field strength etc). A range of values will be understood to include all values within the range, including the values defining the range, and values adjacent to the range which lead to the same or substantially the same outcome as the values immediately adjacent to that value which defines the boundary to the range.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Throughout the specification, the name PMX-205 means the compound cyclo-hydrocinnamate-[Orn-Pro-D-cyclohexylalanine-Trp-Arg] represented by the structure:

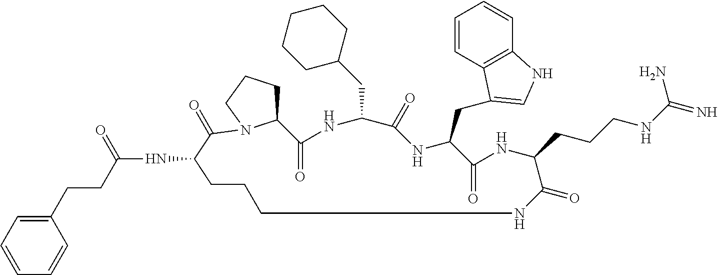 [SMILES: N=C(N)NCCC[C@@H]1NC(=O)[C@H](Cc2c[nH]c3ccccc23)NC(=O)[C@@H](CC2CCCCC2)NC(=O)[C@@H]2CCCN2C(=O)[C@@H](NC(=O)CCc2ccccc2)CCCNC1=O]

Throughout the specification, the name PMX-53 means the compound Ac-Phe-[Orn-Pro-D-Cha-Trp-Arg] represented by the structure:

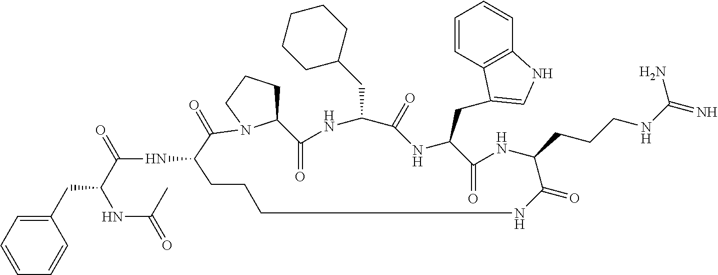 [SMILES: CC(=O)N[C@H](Cc1ccccc1)C(=O)N[C@H]1CCCNC(=O)[C@H](CCCNC(=N)N)NC(=O)[C@H](Cc2c[nH]c3ccccc23)NC(=O)[C@@H](CC2CCCCC2)NC(=O)[C@@H]2CCCN2C1=O]

It must be noted that, as used in the subject specification, the singular forms “a”, “an” and “the” include plural aspects unless the context clearly dictates otherwise.

The term “amino acid side chain” is used in its broadest sense and refers to the side chains of both L- and D-amino acids including the 20 common amino acids such as alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine; and the less common amino acids but known derivatives such as homo-amino acids, N-alkyl amino acids, dehydro amino acids, aromatic amino acids and α,α-disubstituted amino acids, for example, cystine, 5-hydroxylysine, 4-hydroxyproline, α-aminoadipic acid, α-amino-n-butyric acid, 3,4-dihydroxyphenylalanine, homoserine, α-methylserine, ornithine, pipecolic acid, ortho, meta or para-aminobenzoic acid, citrulline, canavanine, norleucine, δ-glutamic acid, aminobutyric acid, L-fluorenylalanine, L-3-benzothienylalanine and thyroxine; and any amino acid having a molecular weight less than about 500.

Common amino acids may be referred to by their full name, standard single-letter notation (IUPAC), or standard three-letter notation for example: A, Ala, alanine; C, Cys, cysteine; D, Asp, aspartic; E, Glu, glutamic acid; F, Phe, phenylalanine; G, Gly, glycine; H, His, histidine; I, Ile isoleucine; K, Lys, lysine; L, Leu, leucine; M, Met, methionine; N, Asn, asparagine; P, Pro, proline; Q, Gln, glutamine; R, Arg, arginine; S, Ser, serine; T, Thr, threonine; V, Val, valine; W, Trp, tryptophan; X, Hyp, hydroxyproline; Y, Tyr, tyrosine. Any and all of the amino acids in the compositions herein can be naturally occurring, synthetic, and derivatives or mimetics thereof.

The term “protected” is used herein in its broadest sense and refers to an introduced functionality which renders a particular functional group, such as a hydroxy, amino, carbonyl or carboxy group, unreactive under selected conditions and which may later be optionally removed to unmask the functional group. A protected amino acid side chain is one in which the reactive substituents of the side chain or the amino group or carbonyl group of the amino acid are protected. Suitable protecting groups are known in the art and include those disclosed in Greene, T. W., “Protective Groups in Organic Synthesis” John Wiley & Sons, New York 1999, (the contents of which are incorporated herein by reference) as are methods for their installation and removal.

The term “alkyl” embraces linear, branched or cyclic radicals having 1 to about 20 carbon atoms, preferably, 1 to about 12 carbon atoms. More preferred alkyl radicals have 1 to about 10 carbon atoms and cycloalkyl radicals have 3 to about 8 carbon atoms. Most preferred are alkyl radicals having 1 to about 6 carbon atoms. Examples of such radicals include methyl, ethyl, n-propyl, isopropyl, n-butyl, isobutyl, sec-butyl, tert-butyl, pentyl, iso-amyl, hexyl and the like. Examples of cycloalkyl radicals include cyclopropyl, cyclobutyl, cyclopentyl and cyclohexyl.

The term “aryl” means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl.

The term “heteroaryl” refers to a 5- or 6-membered substituted or unsubstituted aromatic heterocycle containing one or more heteroatoms selected from N, O and S. Illustrative of such rings are thienyl, furyl, imidazolyl, oxadizolyl, pyridyl or pyrazinyl.

The term “halo” refers to fluorine, chlorine, bromine or iodine.

The term “optionally substituted” means that a group may or may not be further substituted with one or more groups selected from alkyl, alkenyl, alkynyl, aryl, halo, haloalkyl, haloalkenyl, haloalkynyl, haloaryl, hydroxy, alkoxy, alkenyloxy, alkynyloxy, aryloxy, carboxy, benzyloxy, haloalkoxy, haloalkenyloxy, haloalkynyloxy, haloaryloxy, nitro, nitroalkyl, nitroalkenyl, nitroalkynyl, nitroaryl, nitroheterocyclyl, azido, amino, alkylamino, alkenylamino, alkynylamino, arylamino, benzylamino, acyl, alkenyacyl, alkynylacyl, arylacyl, acylamino, acyloxy, aldehydo, alkylsulphonyl, arylsulphonyl, alkysulphonylamino, arylsulphonylamino, alkylsulphonyloxy, arylsulphonyloxy, heterocyclyl, heterocycloxy, heterocyclylamino, haloheterocyclyl, alkylsulphenyl, arylsulphenyl, carboalkoxy, carboaryloxy, mercapto, alkylthio, arylthio, acylthio and the like.

The salts of the compounds of formula A are preferably pharmaceutically acceptable, but it will be appreciated that non-pharmaceutically acceptable salts also fall within the scope of the present invention, since these are useful as intermediates in the preparation of pharmaceutically acceptable salts. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulphuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

The pharmaceutically acceptable acid addition salts of a compound of Formula which contain a basic centre may be prepared in a conventional manner. For example, a solution of the free base may be treated with a suitable acid, either neat or in a suitable solution, and the resulting salt isolated either by filtration or by evaporation under vacuum of the reaction solvent. Pharmaceutically acceptable base addition salts may be obtained in an analogous manner by treating a solution of a compound of Formula A with a suitable base. Both types of salt may be formed or interconverted using ion-exchange resin techniques.

In addition, some of the compounds of Formula A may form solvates with water or common organic solvents. Such solvates are encompassed within the scope of the invention.

By “derivative” is meant any salt, hydrate, protected form, ester, amide, active metabolite, analogue, residue or any other compound which is not biologically or otherwise undesirable and induces the desired pharmacological and/or physiological effect. Preferably the derivative is pharmaceutically acceptable.

The term “tautomer” is used in its broadest sense to include compounds of formula I which are capable of existing in a state of equilibrium between two isomeric forms. Such compounds may differ in the bond connecting two atoms or groups and the position of these atoms or groups in the compound.

The term “isomer” is used in its broadest sense and includes structural, geometric and stereo isomers. As the compound of formula I may have one or more chiral centres, it is capable of existing in enantiomeric forms.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

### BRIEF DESCRIPTION OF THE EMBODIMENTS

Further features of the present invention are more fully described in the following description of several non-limiting embodiments thereof. This description is included solely for the purposes of exemplifying the present invention. It should not be understood as a restriction on the broad summary, disclosure or description of the invention as set out above.

In accordance with one embodiment of the invention, a first synthesis of a compound of Formula A, in the form of PMX-205 as depicted below, is described below.

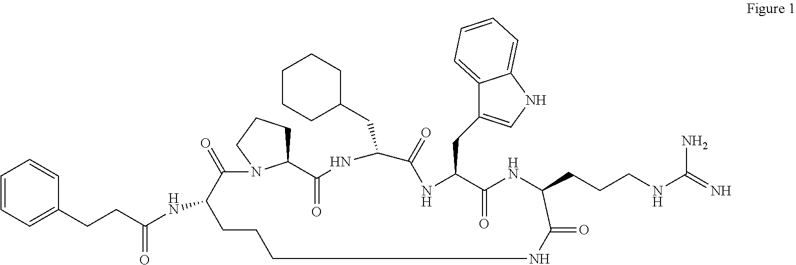 [SMILES: N=C(N)NCCC[C@@H]1NC(=O)[C@H](Cc2c[nH]c3ccccc23)NC(=O)[C@@H](CC2CCCCC2)NC(=O)[C@@H]2CCCN2C(=O)[C@@H](NC(=O)CCc2ccccc2)CCCNC1=O]

Throughout the following description of the Examples, the following abbreviations are used:
AA Amino acid
ACN Acetonitrile
Alloc Allyl alcohol
Boc tert-Butyloxy carbonyl
CV Column volume
DCM Dichloromethane
DCC N,N′-Dicyclohexaylcarbodiimide
DIC N,N′-Diisopropylcarbodiimide
DIPE Diisopropyl ether
DIPEA Diisopropylethylamine
DMAP N,N-Dimethyl-4-pyridinamine
DMF N,N′-Dimethylformamide
DSP Downstream process
eq. Equivalent
HOBt.H2O 1-Hydroxybenzotriazole monohydrate
HPPS Homogeneous phase peptide synthesis
IC Ion chromatography
IPC In-process control
MP Mobile phase
MSI Major single impurity
MTBE Methyl tert-butyl ether
MW Molecular weight
NaDithio Diethyldithiocarbamic acid sodium salt trihydrate
NaPTS Sodium 4-methyl-benzenesulfinate
PyBOP Bbenzotriazol-1-yl-oxytripyrrolidinophosphonium hexafluorophosphate
RCT Reactor
rt Room temperature
SPPS Solid phase peptide synthesis
TFA Trifluoroacetic acid
TIS Triisopropylsilane
Trt Trityl, triphenylmethyl
USP Upstream process

The following raw materials were used in the synthetic methods described below:

| | | |
|---|---|---|
| Name CIS-Number CAS Registry-Nr MOL Formula MOL Weight Abbreviation Supplier | Boc—(Fmoc)Orn—OH n/a 150828-96-9 C₂₅H₃₀N₂O₆ 454.52 Boc—(Fmoc)Orn GL Biochem | 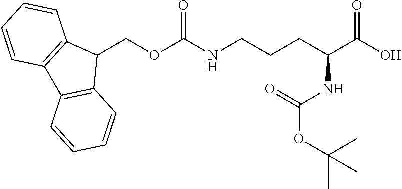 [SMILES: CC(C)(C)OC(=O)N[C@@H](CCCNC(=O)OCC1c2ccccc2-c2ccccc21)C(=O)O] |
| Name CIS-Number CAS Registry-Nr. MOL Formula MOL Weight Abbreviation Supplier | Boc—D—(Fmoc)Orn—OH n/a 163336-15-0 C₂₅H₃₀N₂O₆ 454.52 Boc—D—(Fmoc)Orn GL Biochem | 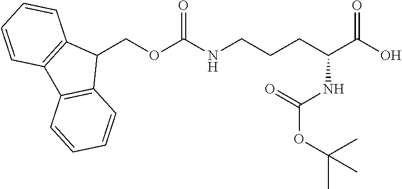 [SMILES: CC(C)(C)OC(=O)N[C@H](CCCNC(=O)OCC1c2ccccc2-c2ccccc21)C(=O)O] |
| Name CIS Number CAS Registry-Nr MOL Formula MOL Weight Abbreviation Supplier | Fmoc—D—Cha—OH 39358 163336-15-0 C₂₄H₂₇NO₄ 393.48 Fmoc—D—Cha GL Biochem | 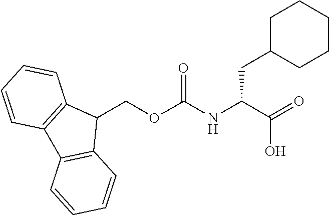 [SMILES: O=C(N[C@H](CC1CCCCC1)C(=O)O)OCC1c2ccccc2-c2ccccc21] |
| Name CIS-Number CAS Registry-Nr. MOL Formula MOL Weight Abbreviation Supplier | Tetrakis(triphenylphosphin) palladium 6605 14221-01-03 C₇₂H₆₀P₄Pd 1155.59 Pd(PPh₃)₄ Energy Chemical | 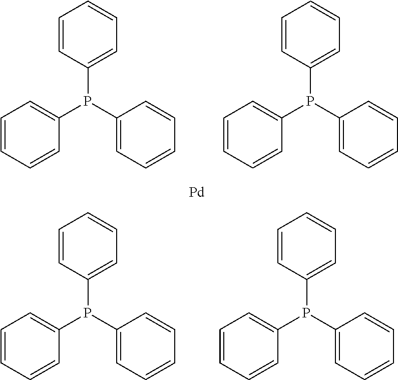 |
| Name CIS-Number CAS Registry-Nr. MOL Formula MOL Weight Abbreviation Supplier | 3 Phenylpropionic hydrochloride 17957 645-45-4 C₉H₉ClO 168.62 H-Cin_Cl Energy Chemical | 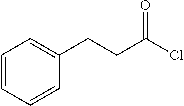 [SMILES: O=C(Cl)CCc1ccccc1] |

### A. Preparation of Boc-Orn(Fmoc)-OAll

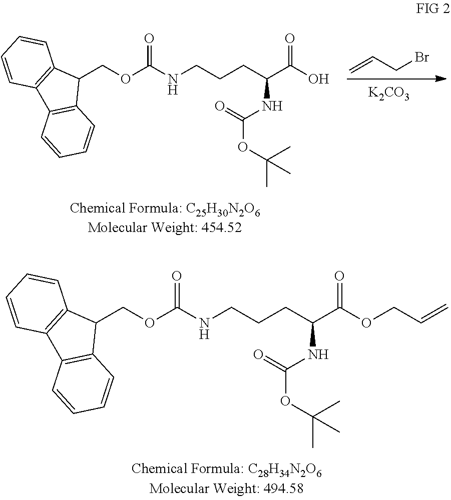

The synthesis started by fully protecting Boc-Orn(Fmoc)-OH with 3-bromopropene/K₂CO₃ instead of the conventional DCC/DMAP strategy for esterification thereby avoiding the formation of an N-acylurea impurity. The product was isolated by precipitation in petroleum ether.

K₂CO₃ (A×0.45, 1.10 eq.) g is added in portions to Boc-Orn(Fmoc)-OH (1.0 eq., A g) in DMF (A×5.0) g solution at 25° C. 0.5 hours later, 3-bromopropene (A×0.57, 3.0 eq.) g is charged dropwise while mass temperature is maintained at 25±5° C. The esterification completion is monitored by HPLC. Solid is removed by filtration.

The filtrate is poured into 10 w % KHSO₄ aqueous solution (A×14.0) g below 0° C. to give rise to a suspension. The suspension is filtered and the wet cake is dissolved with ethyl acetate (A×10.0) g and the resulting organic solution is washed twice with 20% NaCl solution (A×1.2) g each time, in total (A×2.4) g. The organic phase is dried over anhydrous MgSO₄ (A×1.0) g.

After 1 hour, the solid MgSO₄ is removed by filtration and washed twice with ethyl acetate (A×0.5) g each time, in total (A×1.0) g. The filtrate and washes are combined and concentrated below 35° C. in vacuum until about (A×3.0) g of residue remains, followed by addition of petroleum ether (A×10.0) g to precipitate the product. The suspension is cooled to 0° C., filtered and the wet cake is washed with petroleum ether (A×3.0) g and dried in vacuum below 35° C.

### B. Preparation of H-Orn(Fmoc)-OAll

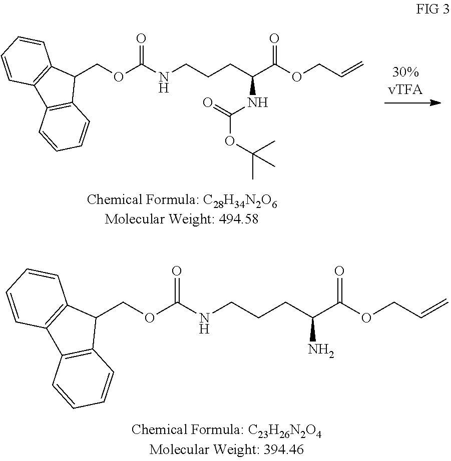 [SMILES: C=CCOC(=O)[C@H](CCCNC(=O)OCC1c2ccccc2-c2ccccc21)NC(=O)OC(C)(C)C ; C=CCOC(=O)[C@@H](N)CCCNC(=O)OCC1c2ccccc2-c2ccccc21]

Removal of the Boc protecting group was conducted in 30 v % TFA in DCM solution. The product was isolated by precipitation in DIPE.

Boc-Orn(Fmoc)-OAll (A, 1.0 eq.) g is dissolved with dichloromethane (A×6.6) g at 20±5° C. To the mixture, TFA (A×7.4) g is charged dropwise while mass temperature is maintained at 20±5° C. The reaction completion is monitored by HPLC.

The reaction mixture is concentrated under reduced pressure to give rise to residue, which then is poured into DIPE (A×10.0) mL, then the suspension is filtered and wet cake is washed three times with DIPE (A×2.0) mL each time, in total A×6.0 g). Wet cake is dried in vacuo below 35° C.

### C. Loading of H-Orn(Fmoc)-OAll on CTC Resin

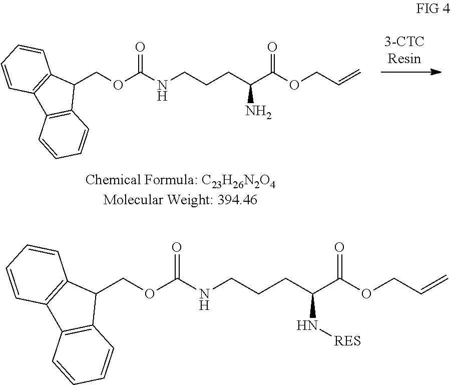

The key intermediate H-Orn(Fmoc)-OAll was introduced readily on 2-chlorotrityl chloride resin in the presence of 5.0 eq. of DIPEA.

2-Chlorotrityl chloride resin (A×2.47, 1.3 eq.) g is swollen for 5 minutes in dry DMF (A×5.0) g at 20±5° C., followed by draining off solvent. Material H-Orn(Fmoc)-OAll (A, 1.0 eq.) g is dissolved with DMF (A×10.0) mL at 20±5° C. To the mixture, DIPEA (A×1.27, 5.0 eq.) g is charged dropwise while the temperature is maintained at 20±5° C. The resulting mixture is transferred to a reaction vessel containing the prepared 2-chlorotrityl chloride resin. The reaction completion is monitored by HPLC.

Once the acceptance criterion is reached (final area % of H-Orn(Fmoc)-OAll in the reaction mixture=3.0% of the start), the resin is drained. A mixed solution of DMF/MeOH/DIPEA=85/10/5 (A×8.0) mL is charged and stirred for 30 minutes with the aim of capping any active point on resin. At final the resin is drained and washed 5 times with DMF (A×10.0) mL for each wash, in total A×50.0) mL.

### D. Assembly of Pentapeptide

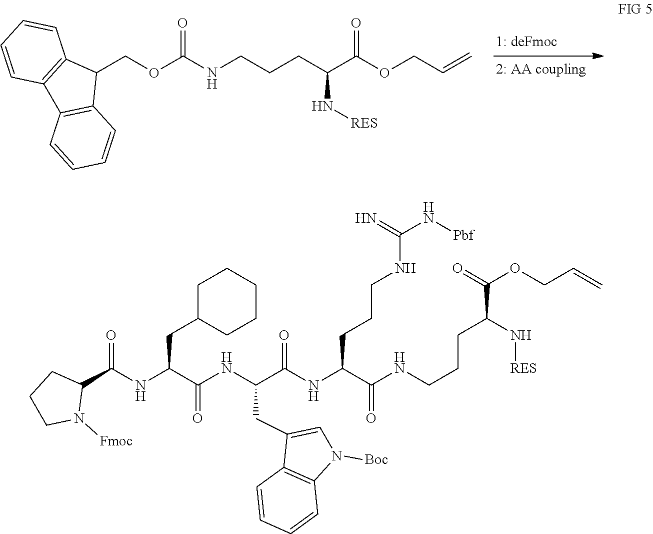

Peptide elongation is carried out on the resin using standard Solid Phase Peptide Synthesis methodology.

To reduce peptide leaching from the peptidyl resin over time, the coupling cycle should be conducted as soon as possible using DMF as solvent.

The deprotection (deFmoc) procedure is as follows:
a. Swell the resin with A×6.5 mL DMF (2 minutes).
b. Perform 2 successive deprotection steps with (A×6.5) mL of a solution of 20% piperidine in DMF (1st cycle for 10 minutes and 2nd cycle for 20 minutes).
c. Wash the resin with A×6.5 mL DMF (6 cycles, 2 minutes of each).

The coupling procedure is as follows:
a. Prepare a solution of mixture of (TA×10⁻³×3.0×MWAA, TA=working scale for the elongation) g of AA and (TA×10⁻³×1×153.1) g of HOBT.H2O in (A×5) mL DMF.
b. Add a portion of (TA×10⁻³×3.0×126.0/0.806) mL of DIC into the transport bottle followed by stirring at room temperature for 2.5 hours.
c. Monitor the reaction progress by means of a Kaiser Test every 30 mins.
d. When the reaction is complete, wash the resin with (A×6.5) mL DMF (3 cycles, 2 minutes of each).

e. If the coupling is not complete, add (TA×10⁻³×0.25×520.4) g of PyBOP (solid) and stir for 1 minute.
f. Adjust the pH of the reaction mixture to pH=7 by means of a solution of 20% DIPEA in DMF and stir for 30 minutes.
g. Control the reaction progress by means of Kaiser test.
h. If positive Kaiser test, repeat the PyBOP treatment.

### E. Selective Deprotection of Allyl Ester

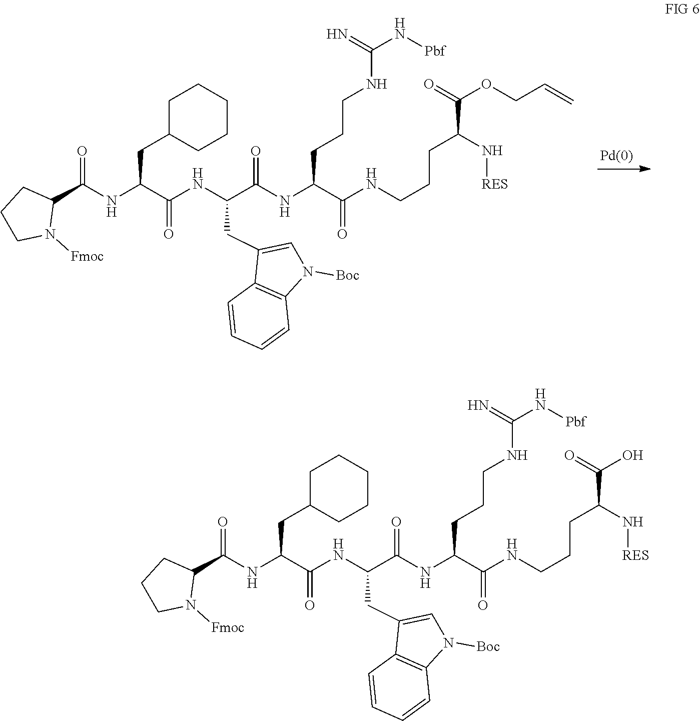

The allyl protecting group was cleaved in the presence of a catalytic amount of Pd(0). Residual Pd was removed ultimately by washing the resin with NaDithio aqueous solution.

The product was prepared as follows:
a. Wash the resin with (A×6.5) mL DMF (4 cycles, 2 minutes for each).
b. Prepare a solution of (TA×10⁻³×2.2×178.2) g of NaPTS in DMF.
c. Add (TA×10⁻³×3.0×98.0/0.85/1.685) g of H₃PO₄ (85 w %) in H₂O to this solution.
d. Let stir until the NaPTS is completely dissolved.
e. Transfer the solution into the reaction vessel.
f. Degas the reaction vessel under stirring with nitrogen bubble (15 minutes).
g. Charge (TA×10⁻³×0.2×1155.6) g of Pd(PPh₃)₄ into the RCT.
h. Pursue the reaction under nitrogen at 50° C. Control the reaction progress by means of HPLC.
i. Wash the resin with (A×6.5) mL DMF (3 cycles, 5 minutes for each).
j. Wash the resin with (A×6.5) mL of 0.02 M (4.51 g/L) solution of NaDithio in 5 v % H₂O in DMF (4 cycles, 5 minutes for each).
k. Wash the resin with (A×6.5) mL DMF (3 cycles, 5 minutes for each).
l. Wash the resin with (A×6.5) mL of a solution of 2 w % HOBt.H2O in DMF (2 cycles, 3 minutes for each).
m. Wash the resin with (A×6.5) mL DMF (2 cycles, 2 minutes for each).

### F. De-Fmoc Reaction

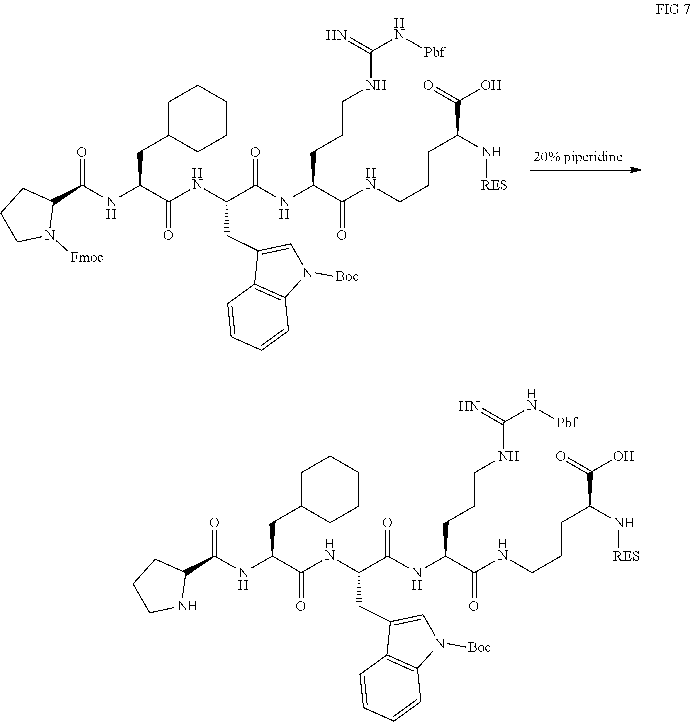

De-Fmoc was conducted with similar procedure, as follows:
a. Swell the resin with (A×6.5) mL DMF (2 minutes).
b. Perform 2 successive de-protection steps with (A×6.5) mL of a solution of 20% piperidine in DMF (1st cycle for 10 minutes and 2nd cycle for 20 minutes).
c. Wash the resin with (A×6.5) mL DMF (6 cycles, 2 minutes for each).

### G. Cyclization on Resin

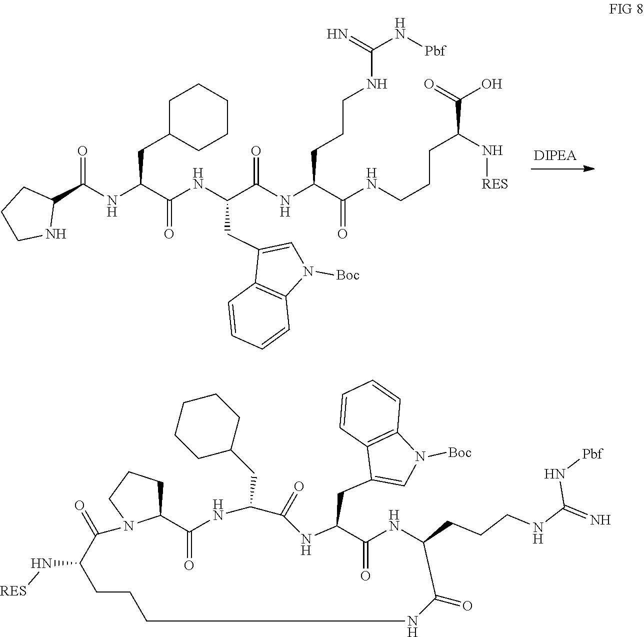

Cyclization between proline and ornithine was performed on the resin directly, as follows:
a. Swell the resin with (A×6.5) mL DMF (2 minutes).
b. Prepare a solution of mixture of (TA×10−3×1.0×328.0) g of TOTU and (TA×10⁻³×1.5×129.24) g of DIPEA in (A×5) mL DMF.
c. Transfer the solution into RCT and stir for 3 hours.
d. Control the reaction progress by means of a Kaiser Test.
e. When the reaction is completed, wash the resin with (A×6.5) mL DMF (3 cycles, 2 minutes of each) and (A×6.5) mL DCM (4 cycles, 2 minutes of each).
f. Dry the peptidyl resin under reduced pressure.
g. If the coupling is incomplete after 3 hours, prepare a solution of mixture of (TA×10−3×0.25×520.4) g of PyBOP in (A×5) mL DMF.
h. Adjust the pH of the reaction mixture to a constant pH=7 with a solution of 20% DIPEA in DMF.

i. Let stir for another 3 hours.

### H. Cleavage from Resin

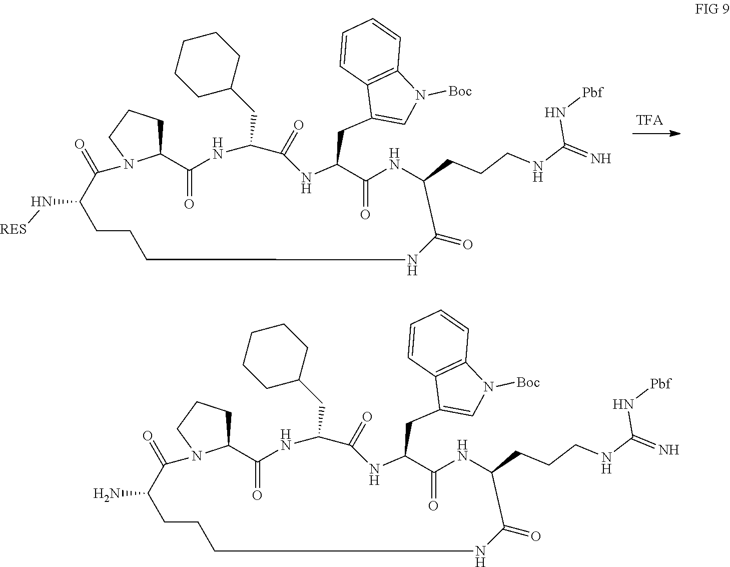

The peptidyl resin was subjected 3 times to 2 v % of TFA in DCM solution to release the protected peptide from resin; after TFA treatment, the combined filtrate solution should be neutralized to pH=7 by DIPEA immediately to reduce any possible decompositions, as follows:
a. Transfer (A×10) mL 2 v % of TFA in DCM solution into RCT.
b. Let stir for 3 minutes.
c. Empty the RCT
d. Adjust pH of filtrate to 7 with 20 v % of DIPEA in DCM solution.
e. Repeat above processes twice.
f. Pool the filtrate mixtures and concentrated it under reduced pressure.
g. Dilute the residue with (A×2) mL DMF.
h. Pour the mixture into (A×15) mL chilled water with stirring.
i. Filter the suspension
j. Wash wet cake twice with (A×3) mL water for each and three times with (A×3) mL DIPE for each.
k. Dry the wet cake under reduced pressure.

### I. Acylation of the N-terminal of Ornithine

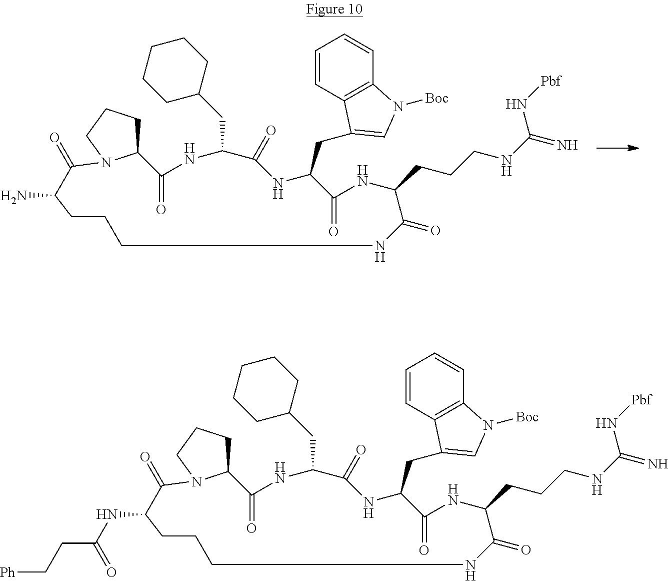

The free amine of the cyclic peptide was acylated with 3-phenylpropionyl chloride in the presence of 3.0 eq. of DIPEA using DCM as reaction solvent; excess of 3-phenylpropionyl chloride and the corresponding hydrolyzed by-product 3-phenylpropionic acid were able to be eliminated by washing with 5 w % NaHCO3 aqueous solution and precipitated in DIPE, as follows.

The cyclic peptide material (1.0 eq. A) g is dissolved in DCM (A×7) mL at 20° C., followed by addition of DIPEA (A×0.37, 3.0 eq.) g. To the resulting solution, the prepared 3-phenylpropionyl chloride (A×0.2, 1.2 eq.) g in DCM (A×mL solution is added dropwise while mass temperature is controlled below 25° C. The reaction progress is controlled by HPLC monitoring.

After completion, the reaction mixture is washed twice with 5 w % of NaHCO3 aqueous solution (A×1.5) mL each time, in total (A×3) mL and 15 w % of brine (A×2) mL. The organic phase is concentrated under reduced pressure until about one fifth of original volume is remained, followed by addition of DIPE (A×10) mL to precipitate the product. The resulting suspension is cooled below 5° C. After 1 hour, the suspension is filtered and the wet cake is washed with DIPE (A×2) mL followed by drying under vacuum below 35° C.

### J. Global De-Protection

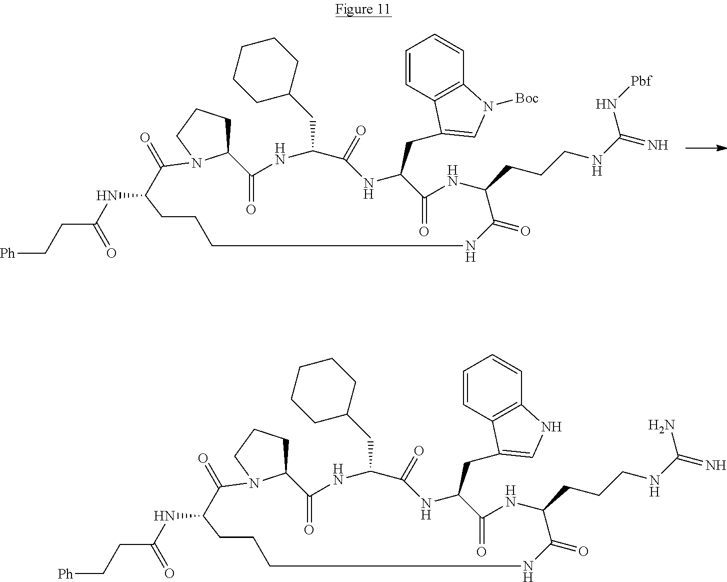

Full de-protection was conducted in a mixture of TFA/TIS/H2O/DODT=90/3/3/4 at 25° C., the reaction mixture was concentrated under reduced pressure, followed by adding di-isopropyl ether to precipitate the crude peptide, as follows.
a. Dissolve the fully protected peptide (A) g in a mixture of TFA/TIS/H2O/DODT=90/3/3/4 (A×5) mL at 0° C.
b. After all the peptide is dissolved, the reaction temperature is allowed to rise to 22° C.
c. HPLC is used to monitor the reaction progress
d. When the reaction is complete, concentrate the organic phase below 35° C. under reduced pressure until about one half of the original volume remains.
e. Addition of DIPE (A×12) mL to precipitate the product at 0° C.
f. Filter the suspension and wash the wet cake three times with DIPE (A×2) mL of each time, in total (A×6) mL.
g. Dry wet cake in vacuo below 35° C.

In accordance with an alternate embodiment of the invention, a second synthesis of a compound of Formula A, in the form of PMX-205, is described below. This synthesis was used to produce larger batches (100 g and 200 g) of the target compound. In the description below, only steps that differ from the preceding discussion of the first synthesis are described.

### K. Selective Deprotection of Allyl Ester

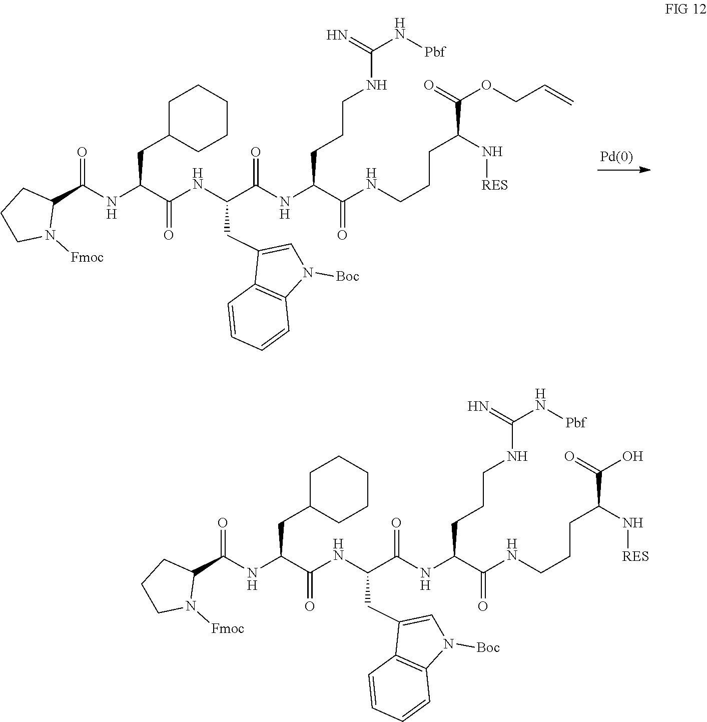

At large scale production, the resulting by-product HPTS precipitated from the system and blocked the embedded filter which caused problematic filtration during washing with NaDithio aqueous solution. It was confirmed that additional water could dissolve the precipitate and ease the filtration process. Accordingly, the step of washing the resin with (A×6.5) mL of 0.02 M (4.51 g/L) solution of NaDithio in 5% H2O in DMF (4 cycles, 5 minutes for each) is replaced with the step of washing the resin with (A×6.5) mL of 0.02 M (4.51 g/L) solution of NaDithio in 50% H2O in DMF (4 cycles, 5 minutes for each).

### L. Cyclization on Resin

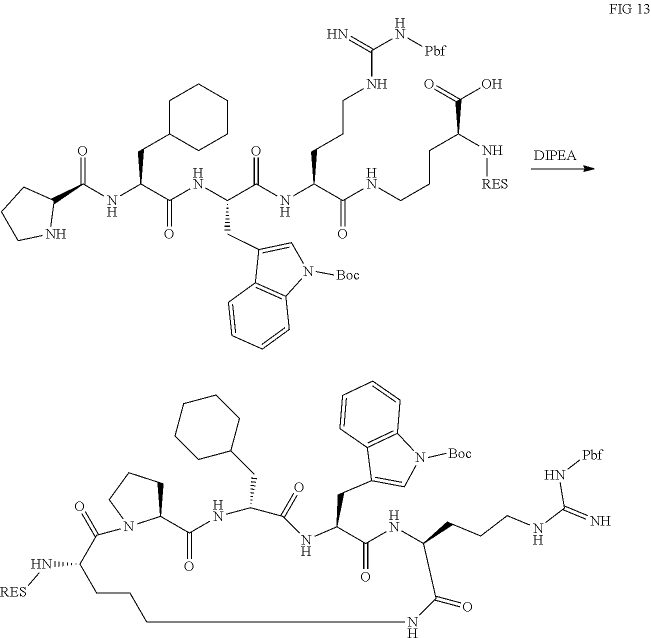

Cyclization on the resin proceeded more quickly in the larger scale reaction. Further, in order to achieve the complete cyclization, addition of a second portion of PyBOP/DIPEA was required. The modified cyclization procedure is as follows:
a. Prepare a solution of (TA×10⁻³×1.0×328.0) g of TOTU and (TA×10⁻³×1.5×129.24) g of DIPEA in (A×5) mL DMF. Transfer the solution into RCT and stir for 2 hours.
b. Empty the RCT.
c. Wash the resin with (A×6.5) mL DMF (2 cycles of 2 minutes each).
d. Prepare a solution of mixture of (TA×10⁻³×0.25×520.4) g of PyBOP and (TA×10⁻³×0.37×129.3) g DIPEA in (A×5) mL DMF. Transfer the solution into RCT and stir for 1 hour.
e. Control the reaction progress by means of a Kaiser Test.

### M. Cleavage from Resin

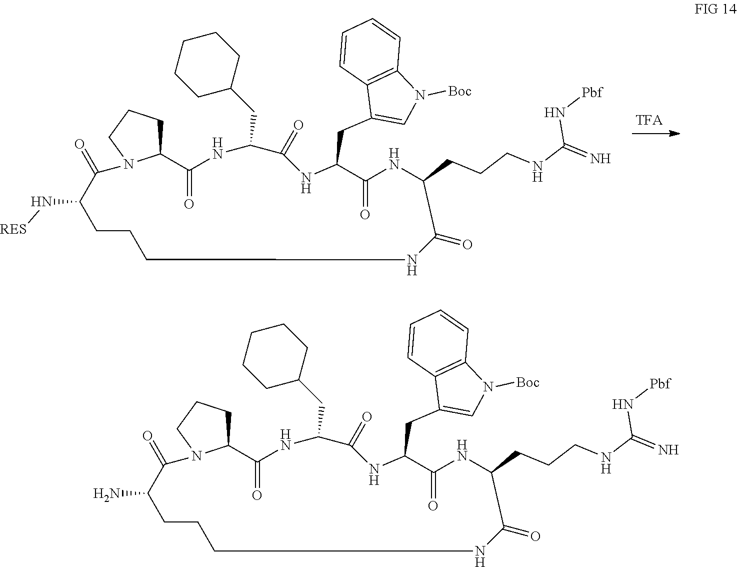

At the larger scale, 2 additional 2 v % TFA treatments were applied to completely cleave the peptide from the resin, such that an improved procedure for larger scales is as follows:
a. Transfer (A×10) mL 2 v % of TFA in DCM solution into RCT.
b. Let stir for 5 minutes.
c. Empty the RCT. Adjust pH of filtrate to 7 with 20 v % of DIPEA in DCM solution.
d. Repeat above processes four times.

Furthermore, in previous small scale experiments, the cleaved peptide was isolated by precipitation in water, followed by washing with MTBE. In some cases, the wet solid was partially dissolved by MTBE and it became too sticky to be filtrated. Further experiments showed that the peptide was soluble in MTBE. As such, for large scale manufacturing, a process was devised in which the isolation process was skipped to avoid the problematic filtration step, as follows:
a. Concentrate the primary DCM solution to ¼ volume, then washed with water (A×1.5) mL.
b. When the aqueous layer is discarded and the organic layer is dried over anhydrous MgSO₄.
c. Remove DCM, the free amine product is obtained as an orange oil, which is used without further purification for the next step.

The improved procedure for workup after selective cleavage is represented in flow-chart form as follows:

While some DIPEA⋅TFA salt was found in the cleaved peptide, it was confirmed that the residual DIPEA⋅TFA had no negative impact on next step and readily removed through preparative HPLC purification. Additionally, no product was found to be lost in aqueous phase.

### N. Crude Yields

The yields for the method of the first embodiment are provided in tabular form below:

| | Weight | Purity |
|---|---|---|
| Compound | (g) | (%) |
| Boc-Orn(Fmoc)-OH | 5.0 | 99.2 |
| Boc-Orn(Fmoc)-OAll | 4.5 | 95.2 |
| H-Orn(Fmoc)-OAll | 4.2 | 98.0 |
| H-[Orn-Pro-D-Cha-Trp(Boc)-Arg(Pbf)] | 4.5 | n/a |
| HCin[Orn-Pro-D-Cha-Trp(Boc)-Arg(Pbf)] | 5.0 | 71.9 |
| Crude HCin-[Orn-Pro-D-Cha-Trp-Arg] | 3.5 | 63.7 |

Thus, the overall USP yield of the preparation of the compound of Formula E is: [(3.5×63.7%)/839.04]/(5.0×99.2%/454.5)=24%.

The method of the second embodiment, at 100 g scale, afforded an unoptimised yield of approximately 36%, and at 200 g scale, an unoptimised yield of approximately 50%.

### O. Purification of Crude Acylated Pentapeptide

The crude peptide was purified by reversed-phase high performance liquid chromatography. Analysis of the purified peptide demonstrated a purity of 99.75% with one single impurity present at a level of 0.25%, as shown in FIG. 14.

Analytical HPLC conditions were as follows:

| HPLC System | Aquity UPLC |
|---|---|
| Column | CSH C18, 21 mm × 150 mm, 1.7 um |
| Mobile Phase | 0.1% TFA in H₂0 |
| | 0.1% TFA in CH₃CN |
| Gradient | 35% to 60% B in 0-25 min |
| | 60% to 85% B in 25-30 min, |
| | 85% to 35% B in 30-30.1 min, |
| | 35% B in 30.1-35 min |
| Column Temperature | 30° C. |
| Detection wavelength | 215 nm/254 nm |
| Flow | 0.3 mL/min |

The single impurity is not the diastereoisomer that would have been obtained if racemisation had occurred during cyclisation. In order to demonstrate this, the diastereoisomer was prepared following the procedure described with the exception that D-Ornithine was used in place of L-Ornithine. It was determined that under the HPLC conditions the D-Ornithine diastereoisomer had a later retention time than the L-Ornithine, as illustrated in FIG. 15.

An HPLC analysis summary of the crude peptide obtained following the described procedure demonstrated the target cyclised peptide (PMX-205) was obtained with 84% purity and was accompanied by less than 1% of the D-ornithine diastereoisomer.

| | Retention | | |
|---|---|---|---|
| Compound | time | Area | Percent |
| PMX205 | 7.422 | 6768295 | 83.76% |
| D-Orn isomer | 7.856 | 79607 | 0.99% |
| of PMX205 | | | |

1. A method for the synthesis of a cyclic ornithine-proline-D-cyclohexylalanine-tryptophan-arginine pentapeptide of Formula A; 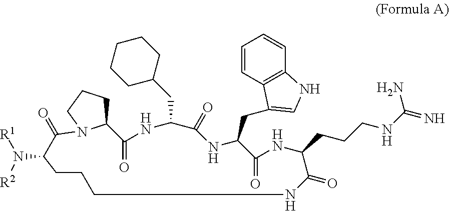
wherein R¹ and R² are, independently, —H, or —(O)R³ where R³ is —CH₂Ph, —CH₂CH₂Ph, —CH═CHPh, —C(NHAC)CH₂Ph;
the method comprising the steps of;
forming a linear proline-D-cyclohexylalanine-tryptophan-arginine-ornithine pentapeptide of Formula B, attached to a polymeric resin; 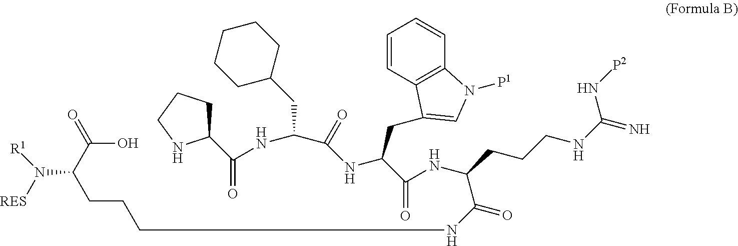
wherein R¹ is as for Formula A, RES indicates the polymeric resin, and P¹ and P² are protecting groups;
cyclising the linear pentapeptide of Formula B to form a cyclic pentapeptide of Formula C, attached to the polymeric resin; 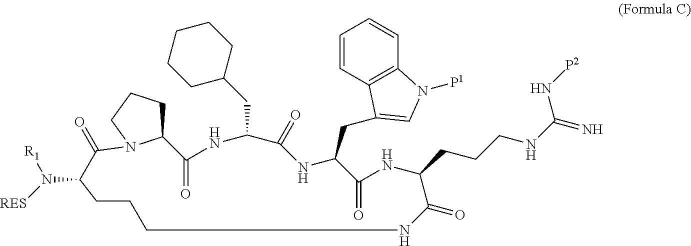
cleaving the cyclic peptide of Formula C from the resin providing a cleaved cyclic pentapeptide having a free amine group of an ornithine residue;
optionally substituting the free amine group of the ornithine residue of the cleaved cyclic peptide;
removing the protecting groups P¹ and P², to provide the cyclic peptide of Formula A.

2. A method according to claim 1 characterised in that the step of cyclising the linear pentapeptide of Formula B to form a cyclic pentapeptide of Formula C, attached to the polymeric resin comprises the steps of treating the linear pentapeptide of Formula B with a combination of a coupling agent and a base, wherein the combination of coupling agent and base is selected from the group: O-[(ethoxycarbonyl)cyanomethylenamino]-N,N,N′,N′-tetramethyluronium tetrafluoroborate (TOTU) and diisopropylethylamine (DIPEA) in dimethylformamide, and benzotriazol-1-yl-oxytripyrrolidinophosphonium hexafluorophosphate (PyBOP) and diisopropylethylamine (DIPEA) in dimethylformamide.

3. A method according to claim 1 characterised in that the step of forming a linear peptide of Formula B, attached to a polymeric resin;
comprises the step of forming the intermediate compound of Formula D; 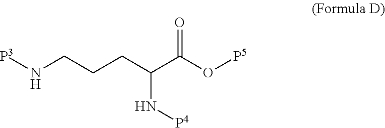
wherein P³, P⁴ and P5 are protecting groups.

4. A method according to claim 3 characterised in that P³ and P⁴ are, independently, selected from the group: 9-fluorenylmethyl carbamate (Fmoc), 2,2,2-trichloroethyl carbamate (Troc), t-butyl carbamate (Boc), allyl carbamate (Alloc), 2-trimethylsilylethyl (Teoc) and benzyl carbamate (Cbz); and P⁵ is selected from the group: -Me, -Et, -tBu, -Bz, —CH₂CH═CH (Alloc).

5. A method according to claim 4 characterised in that P³ and P⁴ are, independently, selected from the group: Fmoc and Boc.

6. A method according to claim 3 characterised in that the compound of Formula D is produced by reacting a compound of Formula E: 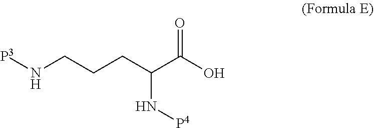
with a compound of the formula P⁵-X, where X is a halide or an alcohol.

7. A method according to claim 3 characterised in that the method comprises the step of coupling the compound of Formula D to a polymeric resin to produce a compound of Formula F:

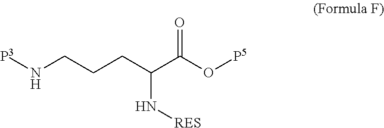

8. A method according to claim 7 characterised in that P⁵ is Alloc.

9. A method according to claim 7 characterised in that P³ is Fmoc.

10. A method according to claim 8 characterised in that the compound of Formula D is produced by reacting a compound of Formula E with a compound of the formula X—CH₂CH═CH.

11. A method according to claim 10 characterised in that the compound of formula X—CH₂CH═CH is 3-bromopropene.

12. A method according to claim 3 characterised in that after the steps of producing the Compound of Formula D, and coupling the Compound of Formula D to the polymeric resin to produce the compound of Formula F, the method comprises the step of sequentially coupling the following amino acids commencing at the distal amine of ornithine;
1) arginine;
2) tryptophan;
3) D-cyclohexylalanine; and
4) proline;
to produce the linear pentapeptide of Formula B.

13. A method according to claim 1 characterised in that the method produces in excess of 200 g of the compound of Formula A.

14. A compound of Formula A produced by the method of claim 1.

15. A pharmaceutical composition comprising a compound of Formula A produced by the method of claim 1.
